(19) United States
(12) Patent Application Publication
(10) Pub. No.: US 2026/0308469 A1
(43) Pub. Date: Oct. 8, 2026

(54) DRY GRIND SYSTEM AND METHOD WITH FRONT END GERM SEPARATION FOR PRODUCING ONE OR MORE SUGAR STREAMS

(71) Applicant: Fluid Quip Technologies, LLC, Cedar Rapids, IA (US)

(72) Inventors: David Koziol, Fort Calhoun, NE (US); Christopher Kempf, Cedar Rapids, IA (US)

(21) Appl. No.: 19/639,380

(22) Filed: Apr. 6, 2026

Related U.S. Application Data
(60) Provisional application No. 63/784,386, filed on Apr. 7, 2025.

(51) Int. Cl. C10L 1/18; C10B 57/06; C10G 69/04

(52) U.S. Cl. CPC C10L 1/1817; C10B 57/06; C10G 69/04; C10G 2300/1014; C10L 2200/0484

(57) ABSTRACT

Improved dry grind systems and methods with front end germ separation for producing one or more sugar (or carbohydrate) streams for biochemical production, such as alcohol. Corn germ, for example, can contain high concentrations of oil and ash, and other impurities that can have negative impacts to unit operations in a dry grind plant. Front end germ separation from a corn flour slurry can provide desirable improvements to dry grind systems and methods. The separated germ/germ fragments can be further subjected to additional processing separate and apart from the further processing of the stream from which the germ was removed, which can produce a more desirable sugar (or carbohydrate) stream due to the reduced amount of undesirable, unfermentable components (e.g., reduced amount of germ) and which may be subjected to liquefaction and eventually fermentation (or other process(es)) such as for biochemical production.

### TECHNICAL FIELD

The present invention relates generally to systems and methods for use in the biochemical (e.g., biofuel), food, feed, nutrition, enzymes, amino acids, proteins, and/or pharmacy industries and, more specifically, to improved dry grind systems and methods with front end germ separation for producing one or more sugar streams, such as for biochemical production.

### BACKGROUND

The majority of fuel alcohol (i.e., ethanol) in the United States is produced from No. 2 Yellow Dent Feed Corn. The most common method of production is the dry grind ethanol process, which is illustrated in FIG. 1. Traditionally, dry milling ethanol plants generally convert corn and/or other grains into three products, i.e., ethanol, distillers grain oil, and distiller's grains with solubles. A typical grain dry milling process consists of four major steps: grain handling and dry milling, liquefaction and saccharification, fermentation and distillation, and co-product recovery. Grain handling and dry milling is the step in which the grain is brought into the plant and ground to promote better conversion of starch to glucose. Liquefaction is the step of converting solids, such as starch, to a flowable liquid producing oligosaccharides and saccharification is where the oligosaccharides are converted into single glucose molecules. Fermentation is the process of yeast or bacteria, or as clostridia, for example, converting glucose into a biofuel or a biochemical/biomolecule, such as ethanol. Distillation is the process of removing the biofuel or biochemical/biomolecule, such as ethanol, from the fermentation product. Co-product recovery is the step in which the grain by-products are de-watered and made ready for market. There are many known chemical and biological conversion processes known in the art that utilize yeast, bacteria, or the like to convert glucose/sugar to other biofuels and biochemical/biomolecule components like ethanol, for example.

In today's typical grain to biochemical plants (e.g., corn to alcohol plants), many systems, particularly dry grind systems, process the entire corn kernel through fermentation and distillation. Such designs require about 30% more front-end system capacity because there is only about 70% starch in corn, with less for other grains and/or biomass materials. Additionally, extensive capital and operational costs are necessary to process the remaining non-fermentable components within the process. By removing undesirable, unfermentable components prior to fermentation (or other reaction process), more biochemical, biofuel, and other processes become economically desirable.

Accordingly, it would be beneficial to provide a system and method for biochemical production that can reduce the amount of undesirable, unfermentable components prior to fermentation (or other reaction process(es)), thereby helping to keep overall capital and operational costs at a desirable level for the dry grind plant and producing more desirable sugar streams, such as for fermentation or other reaction processes.

### SUMMARY OF THE INVENTION

The present invention is directed to a dry grind system and method with front end germ separation for producing one or more sugar (or carbohydrate) streams, such as for biochemical production.

By relying on the densities of the various components of a corn kernel, ground corn flour or broken corn kernels can be separated by different means or methods into different layers or components in a liquid slurry/solution, such as by naturally settling into components generally in layers in a tank or by being mechanically separated. Notably, corn germ contains the most concentrated oil and is of a lower density than the rest of the corn kernel, which can allow for separation of the germ within and from a slurry mixture. Other lighter corn constituents such as stalks, damaged corn, and red dog also have a tendency to float or have higher buoyancy in a liquid slurry/solution, which aids in the overall ability to separate out said components. Separating these lower density constituents in the corn flour can benefit downstream processing by removing unwanted/undesirable, unfermentable impurities including, but not limited to, oil, ash, and minerals.

Corn germ, for example, can contain high concentrations of oil, ash, and minerals (soluble and insoluble), and other impurities that can have negative impacts to unit operations in a dry grind plant. As such, early front end germ separation can provide desirable improvements to dry grind systems and methods. The germ/germ fragments can be further subjected to additional processing separate and apart from the further processing of the slurry stream from which the germ was removed. The degermed stream can produce a more desirable sugar (or carbohydrate) stream due to the reduced amount of undesirable, unfermentable components (e.g., reduced amount of germ) and which may be subjected to liquefaction and eventually fermentation (or other process(es)) such as for biochemical production. In one example, the germ/germ fragments can be further subjected to liquefaction and eventually fermentation (or other process(es)) such as for biochemical production, separate and apart from the further processing of the stream from which the germ was removed (i.e., from the degermed stream). In another example, the separated germ can be further optionally processed in an effort to recover additional oil contained therein so as to increase oil yields. Ultimately, due to the removal of the germ, the resulting sugar (or carbohydrate) stream from the degermed stream can include a desirable decrease in fructose and ash and a desirable increase in dextrose.

In one embodiment, a method with front end germ separation for producing a sugar stream is provided and includes mixing ground grain particles derived from a feedstock of grain and/or grain components with a liquid within a settling tank to produce a slurry that comprises starch, fiber, and germ particles followed by, via weights, separating out lighter germ particles from the slurry in the settling tank to degerm the slurry and allowing heavier solids within the slurry to settle out within the settling tank. Next, a heavier first portion of the settled solids is removed from the settling tank followed by optionally removing a lighter second portion of the settled solids. The heavier first portion and the optional lighter second portion is subjected to liquefaction to provide a liquefied starch solution, which includes starch and fiber, and then the liquefied starch solution is subjected to saccharification to convert the starch to simple sugars thereby producing a sugar stream.

In another embodiment, a method with front end germ separation for producing a sugar stream is provided and includes mixing ground grain particles derived from a feedstock of grain and/or grain components with a liquid within a settling tank to produce a slurry that comprises starch, fiber, and germ particles followed by, via weights, separating out lighter germ particles from the slurry in the settling tank to degerm the slurry and allowing heavier solids within the slurry to settle out within the settling tank. Next, a defined amount of mass/volume of the settled solids is removed from the settling tank to provide a heavier first portion followed by removing a defined amount of mass/volume of remaining settled solids from the settling tank to provide a lighter second portion. The heavier first portion is subjected to a first liquefaction and the lighter second portion is separately subjected to a second liquefaction to provide a first and a second liquefied starch solution, respectively, which include starch and fiber. And then, the first liquefied starch solution is subjected to a first saccharification and the second liquefied starch solution separately subjected to a second saccharification to convert the starch to simple sugars in the first and second liquefied starch solutions thereby producing a first sugar stream and a second sugar stream.

In another embodiment, a system with front end germ separation for producing a sugar stream is provided and includes a settling tank within which ground grain particles derived from a feedstock of grain and/or grain components mix with a liquid to produce a slurry that comprises starch, fiber, and germ particles. The settling tank is configured to separate out lighter germ particles, via weights, from the slurry to degerm the slurry, and in which heavier solids within the slurry settle out. The settling tank is further configured to remove a heavier first portion of the settled solids followed by an optional lighter second portion of the settled solids from the settling tank. A liquefaction system receives the heavier first portion and the optional lighter second portion to provide a liquefied starch solution, which includes starch and fiber, and whereat the starch begins to convert to oligosaccharides. A saccharification system is situated after the liquefaction system and receives the liquefied starch solution. The saccharification system converts the oligosaccharides to simple sugars thereby producing a sugar stream.

The features and objectives of the present invention will become more readily apparent from the following Detailed Description taken in conjunction with the accompanying drawings.

### BRIEF DESCRIPTION OF THE DRAWINGS

The accompanying drawings, which are incorporated in and constitute a part of this specification illustrate embodiments of the invention and, together with the general description of the invention given above and the detailed description given below, serve to explain the principles of the invention. Similar reference numerals are used to indicate similar features throughout the various figures of the drawings.

FIG. 1 is a flow diagram of a typical dry grind alcohol production process;

FIG. 2 is a flow diagram of a dry grind system and method for biochemical production with front end germ separation for producing one or more sugar streams, such as for biochemical production, in accordance with an embodiment of the invention;

FIG. 3 is a detailed view of a portion of the flow diagram of FIG. 2 showing primarily the settling tank 102 and operation thereof, including interplay with the other areas of the dry grind system and method for biochemical production with front end germ separation for producing one or more sugar streams; and

FIG. 4 is a flow diagram of a dry grind system and method for biochemical production with front end germ separation for producing one or more sugar streams, such as for biochemical production, in accordance with another embodiment of the invention.

### DETAILED DESCRIPTION OF SPECIFIC EMBODIMENTS

The present invention relates to dry grind systems and methods with front end germ separation for producing one or more sugar (or carbohydrate) streams for biochemical, such as alcohol (e.g., ethanol), in which germ and/or germ fragments are separated from the initial slurry stream after combining milled feedstock (e.g., milled corn) with liquid to produce a slurry of ground corn (corn flour) and water. After separation of the germ and/or germ fragments, the resulting slurry can be used to produce a more desirable sugar (or carbohydrate) stream due to the reduced amount of undesirable, unfermentable components (e.g., reduced amount of germ/germ fragments), which can include undesirable oil and ash, for example. The degermed stream can be subjected to further processing including liquefaction and eventually fermentation (or other process(es)) such as for producing biochemicals (that can be utilized in food products and pharmaceuticals, for example), as further discussed in detail below.

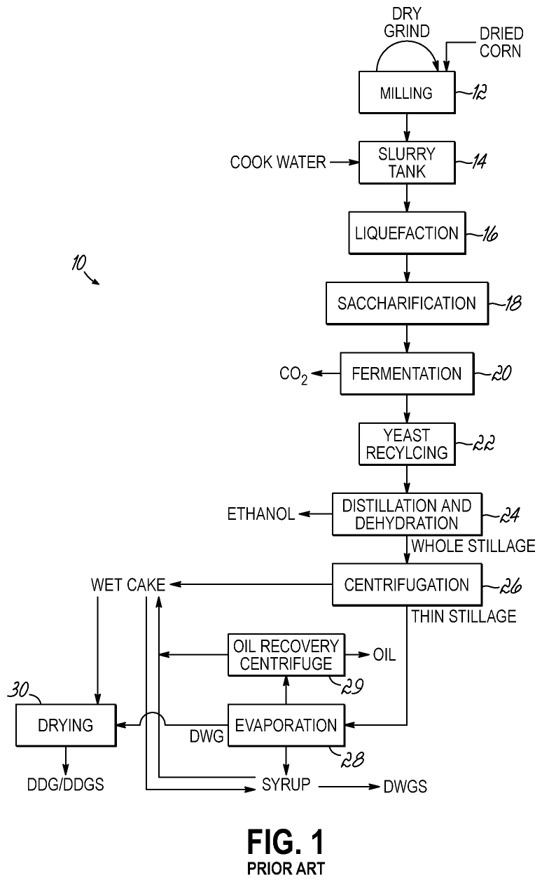

With reference now to FIG. 1, this figure shows a flow diagram of a typical dry grind alcohol (e.g., ethanol) production system and method 10. Although virtually any type and quality of grain, such as but not limited to sorghum, wheat, triticale, barley, rye, tapioca, cassava, potato, pea and other starch and/or oil containing grains and/or legumes can be used to produce ethanol and/or a biochemicals/biomolecules, for example, the feedstock for this process is typically corn referred to as “No. 2 Yellow Dent Corn”. Also, as a general reference point, the dry grind method 10 can be divided into a front end and a back end. The part of the system and method 10 that occurs prior to and including distillation 24 can be considered the “front end,” and the part of the system and method 10 that occurs after distillation 24 can be considered the “back end.”

The front end of the dry grind system and method 10 begins with a milling step 12 in which whole corn kernels can be passed through hammer mills for grinding/milling into meal or a fine powder. The screen openings in the hammer mills or similar devices typically are of a size 6/64 to 9/64 inch, or about 2.38 mm to 3.57 mm, but some plants can operate at less than or greater than these screen sizes. The resulting particle distribution yields a very widely spread, bell type curve, which includes particle sizes as small as 45 microns and as large as 2 mm to 3 mm. The majority of the particles tend to be in the range of 500 to 1200 microns, which is the “peak” of the bell curve. Other screen openings larger and smaller can be deployed in the hammer mills. Other milling devices such as roller mills or pin mills can also be utilized in the dry grain grinding step.

After the milling step 12, the ground meal is mixed with cook water to create a slurry at the slurry tank 14 and a commercial enzyme called alpha-amylase is typically added (not shown). Creating the slurry at the slurry tank 14 is typically followed by a liquefaction step 16 whereat the pH may be adjusted to about 4.8 to 5.8 and the temperature maintained between about 50° C. to 105° C. so as to convert the insoluble starch in the slurry to soluble starch. The stream after the liquefaction step 16 has about 30% dry solids (DS) content, but can range from about 29-36%, with all the components contained in the corn kernels, including starch/sugars, protein, fiber, starch, germ, grit, oil, ash, and salts, for example. Higher solids are achievable, but this requires extensive alpha amylase enzyme to rapidly breakdown the viscosity in the initial liquefaction step. There generally are several types of solids in the liquefaction stream: fiber, germ, and grit.

Liquefaction 16 may be followed by separate saccharification and fermentation steps, 18 and 20, respectively, although in most commercial dry grind ethanol processes, saccharification and fermentation can occur simultaneously. This single step is referred to in the industry as “Simultaneous Saccharification and Fermentation” (SSF). Both saccharification and SSF can take as long as about 50 to 60 hours (or greater). In the saccharification step 18, the liquefied mash is cooled and a commercial enzyme, such as gluco-amylase, can be added to hydrolyze the maltodextrins and short-chained oligosaccharides into single glucose sugar molecules. In the fermentation step 20, a common strain of yeast (Saccharomyces cerevisiae) can be added to metabolize the glucose sugars into ethanol and CO₂. Other fermentation agents such as bacteria and clostridia can be utilized to produce other biomolecules/biochemicals. Upon completion, the fermentation mash (“beer”) will contain about 17% to 18% ethanol (volume/volume basis), plus soluble and insoluble solids from all the remaining grain components, including fiber, protein, and oil, for example. Yeast can optionally be recycled in a yeast recycling step 22 either during the fermentation process or at the very end of the fermentation process. Yeast produced during the fermentation process will pass through to the distillation and dehydration step 24. In addition to the gluco-amylase being added, other enzymes can be added (such as but not limited to phytase, protease, cellulase, hemicellulose, xylanase, beta-glucanase, and the like) that can further enhance the protein and oil recovery downstream. Subsequent to the fermentation step 20 is the distillation (and dehydration) step 24, which utilizes a still to recover the alcohol (e.g., ethanol).

Finally, on the back end, a centrifugation step 26 involves centrifuging the residuals, i.e., “whole stillage”, which includes the non-fermentable grain components (protein, oil, fiber, ash, and minerals, for example) and yeast yielded from the distillation and dehydration step 24 in order to separate the insoluble solids (“wet cake”) from the liquid (“thin stillage”). The liquid from the centrifuge contains about 5% to 12% DS. The “wet cake” includes fiber, of which there generally are three types: (1) pericarp, with an average particle size typically about 1 mm to 3 mm; (2) tipcap, with an average particle size about 500 micron; (3) and fine fiber, with an average particle size of about 250 microns. There may also be proteins and yeast bodies with a particle size of about 45 microns to about 300 microns. The fiber and other fractions may contain bound protein that is chemically and or physically attached to the fiber and other fraction.

The thin stillage typically enters evaporators in an evaporation step 28 in order to boil or flash away moisture, leaving a thick syrup which contains the soluble (dissolved) solids (mainly protein and starches/sugars) from the fermentation (25 to 40% dry solids) along with residual oil and fine fiber. The concentrated slurry can be sent to a centrifuge to separate the oil from the syrup in an oil recovery step 29. The oil can be sold as a separate high value product. The oil yield is normally about 0.9 lb/bu of corn with elevated free fatty acids content compared to traditional wet mill corn oil. This oil yield recovers only about ½ of the oil in the corn, with part of the oil passing with the syrup stream and the remainder being lost with the fiber/wet cake stream. About one-half of the oil inside the corn kernel remains inside the germ after the distillation and dehydration step 24, which cannot be separated in the typical dry grind process using centrifuges as the oil is bound, not free. The free fatty acids content, which is created when the oil is heated and exposed to oxygen throughout the front and back-end process, reduces the value of the oil. The (de-oil) centrifuge only removes less than 50% because the protein and oil make an emulsion, which cannot be satisfactorily separated without the use of chemicals or added mechanical separation unit operations.

The syrup, which has more than 10% oil, can be mixed with the centrifuged wet cake, and the mixture may be sold to beef and dairy feedlots as Distillers Wet Grain with Solubles (DWGS). Alternatively, the wet cake and concentrated syrup mixture may be dried in a drying step 30 and sold as Distillers Dried Grain with Solubles (DDGS) to dairy and beef feedlots. This DDGS has all the corn and yeast protein and about 50% of the oil in the starting corn material. But the value of DDGS is low due to the high percentage of fiber, and in some cases the oil is a hindrance to animal digestion and lactating cow milk quality.

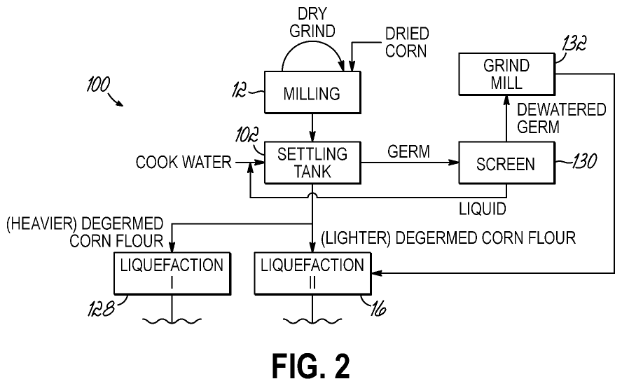

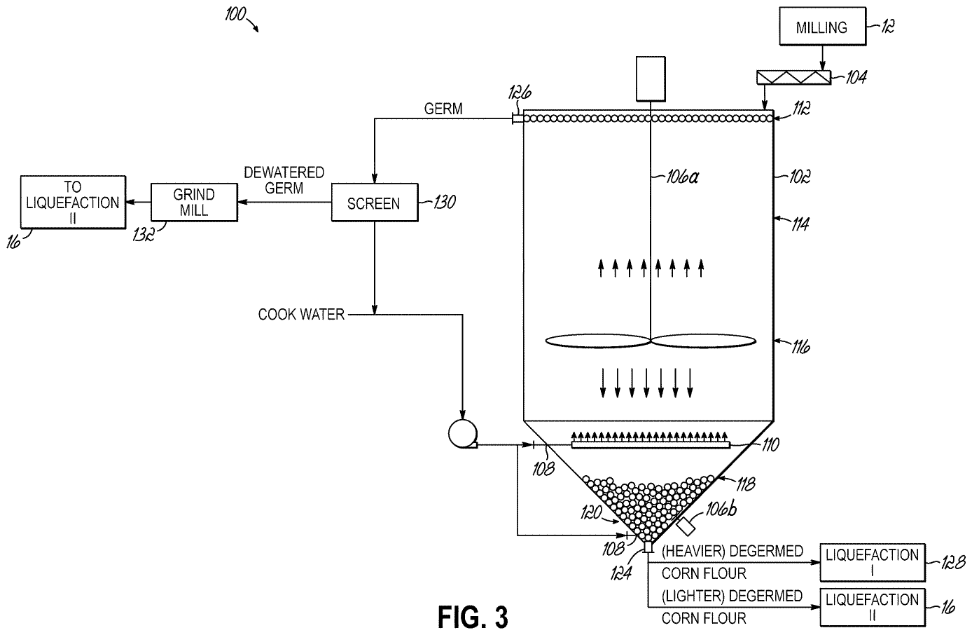

In accordance with the present invention, FIGS. 2 and 3 show an embodiment of a dry grind system and method with front end germ separation for producing/ultimately producing one or more sugar streams, for example, such as for biochemical production (e.g., alcohol), collectively numeral 100, generally based on the dry grind process 10 shown in FIG. 1 and described hereinabove, with modifications/improvements made thereto. In one example, the sugar (or carbohydrate) stream(s) may be produced offsite. In another example, a carbohydrate stream(s) initially may be produced after front end germ separation such as for biochemical production. In another example, a carbohydrate stream(s) initially may be produced after front end germ removal followed by production of a sugar stream(s) such as for biochemical production. This dry grind system and method 100 can reduce the amount of undesirable, unfermentable components, such as germ/germ fragments, prior to fermentation (or other reaction process), thereby helping to keep overall capital and operational costs at a desirable level for the dry grind plant and producing a more desirable sugar (or carbohydrate) stream, such as for fermentation or other reaction processes or utilized directly as an ingredient (such as in a food product or pharmaceutical, for example). The details of the modifications to FIG. 1 as set out in the embodiment of FIGS. 2 and 3 are discussed hereinbelow. It is noted that certain reference numerals used in FIG. 1 are used here to represent like devices and/or steps in the method and system 100 of FIGS. 2 and 3.

As shown in FIGS. 2 and 3, in this embodiment, grain (e.g., corn) feedstock, which may be dried, initially can be milled at milling step 12, like discussed above, with the ground meal (corn flour) being mixed with liquid, such as cook water, at a settling tank 102 to create a slurry. In one example, the cook water can be backset and/or fresh (or clean) water and can be piped in by means and methods known in the art, such as via a pump or other methods and the like. In one example, the temperature of the liquid (e.g., cook water) can be from about 60° F. to about 220° F. In another example, the temperature of the liquid (e.g., cook water) can be from about 110° F. to about 150° F. In another example, the temperature of the liquid (e.g., cook water) can be about 130° F.

As best shown in FIG. 3, the ground feedstock (e.g., corn flour) can be transported by means and methods known in the art, such as via a conveyor 104 and the like, to the settling tank 102. The settling tank 102 can be generally cylindrical in shape with a conical bottom portion (although various other shapes are contemplated and known in the art) and can be equipped with at least one primary agitator 106a, which can include one or more rotating fans/paddles or blades (two fans/blades shown here), controlled by a motor as known in the art. In one example, the agitator 106a can define a screw such as a helical screw. It should be understood that more than one agitator 106a may be utilized and that the agitator 106a can include one or more than two fans/paddles or blades, as desired/needed, and can be of any desired shape or size. The agitator 106a can be stopped and started, as desired, to mix (e.g., homogenously mix) the ground feedstock and liquid, e.g., cook water, to a desired or targeted dry solids (DS) level such as where the slurry is pumpable and still able to be agitated. In one example, the desired DS of the slurry in the settling tank 102 can be from about 30% to 36%. In another example, the desired DS of the slurry can be from about 32% to 34. For example, with respect to the DS level, the slurry should be thin enough (low enough DS) to allow for the classification/self-separation of the components in the settling tank 102 but not so thin (too low) that the water balance is negatively impacted. Mixing of the ground feedstock with the liquid in the settling tank 102 can occur in batches or a semi-continuous type manner. In one example, the settling tank 102 can be operated on level control, increasing or decreasing the amount of material moved or pumped out of the settling tank 102 to match the amount of material entering, by means and methods known in the art. Settling of the slurry mixture into its respective components/layers is allowed to occur after a desired amount of mixing time, as further discussed below.

With continuing reference to FIG. 3, the settling tank 102 can include one or more liquid inlets 108 that communicate with an interior of the settling tank 102 to fill the tank with liquid (e.g., wash water). In one embodiment, one of the liquid inlets 108 can communicate with a water diffuser/distribution header 110, which can be generally situated in a location spaced apart from the bottom of the settling tank 102. In one example, the water diffuser 110 can be adapted/configured to disperse the incoming liquid in a generally even manner within the settling tank 102 to promote mixing and promote an upward flow, as generally indicated by the arrows from the water diffuser 110. The other liquid inlet 108 can be situated at or near the bottom of the settling tank 102, such as within the conical portion, to bring liquid in through the bottom, which can cooperate with a secondary agitator 106b, to aid in mixing of heavier, accumulated ground feedstock near the bottom of the settling tank 102 and to help prevent bridging of the bottom materials. One or more enzymes can be added to the slurry here. In one example, the one or more enzymes can include amylase, alpha-amylase, glucoamylase, fungal, phytase, protease (e.g., serine protease), cellobiose, cellulase, hemicellulase, xylanase, glucanase, beta-glucanase, transglutaminase (e.g. microbial), and the like.

After the ground flour has been delivered to the settling tank 102, agitation can continue for a desired or designated period of time so that the slurry has been sufficiently mixed or homogenized and for the classification of the components. In one example, total mixing time via the agitators 106a and/or 106b can be from about 30 seconds to about 60 minutes. In another example, mixing via the agitators 106a and/or 106b can occur for about 5 minutes to about 20 minutes. During mixing, lighter (or less dense) germ/germ fragments and other lighter or fine particles (e.g., red dog and free oil) can migrate toward the upper portion or top of the slurry mixture/top of the settling tank 102 while larger and more dense material/particles (e.g., starch, protein, and fiber) can have a tendency to sink towards or remain at or near the bottom of the slurry/bottom of the settling tank 102, as indicated generally by the up and down arrows in the settling tank 102. After the pre-determined/desired amount of time, agitation can be stopped and the mixture can be allowed to settle more fully into its classified state. That is, by utilizing the densities of the various components of the ground feedstock (e.g., corn flour), the corn flour can be separated relatively effectively into different layers or portions in the slurry. It should be understood that the layers may not be fully discrete insofar as one or more layers may overlap or mix/blend into one another along general boundary lines. In one example, the slurry mixture can be allowed to settle for about 30 seconds to about 60 minutes. In another example, the slurry mixture can be allowed to settle for about 1 minute to about 10 minutes. Although not shown, additional aides to assist with classification of the various components in the settling tank 102 can be used and can include, for example, sonication or ultrasound systems/devices, compressed and diffused air, chemical and enzyme aids, including surfactants and salts, or other chemical agents that can desirably impact the ionic strength of the slurry mixture, and the like.

The settled slurry mixture now can define or include multiple general and/or specific layers. With reference to FIG. 3, in one embodiment, the settled slurry mixture can include an upper or top/topmost portion or layer having undesirable, unfermentable components, such as germ and/or germ fragments (e.g., fine germ), red dog (red fibrous material), stalks, and other undesirable/unfermentable components, indicated generally by reference numeral 112. Moving from the top towards the bottom, a next portion or layer can generally include a solubles layer, such as protein, starch, sugars/carbohydrates, and the like, as indicated generally by reference numeral 114. Next, below the solubles layer or portion is a layer or portion of (fine) starch and larger fiber or fibrous particles/fragments, which typically have bound starch and protein, as indicated generally by reference numeral 116. Although the components in these three layers can be considered valuable corn constituents, the solubles and germ fragments can be detrimental to multiple unit operations in a biochemical process, such as due to the oil, ash, and resulting fructose contained therein. The portions or layers of the slurry mixture can shift (e.g., increase in size/overlap or move upwards/downwards in order) and the materials in each layer can potentially vary due to a number of factors, such as temperature, mineral or ionic strengths, chemical additions (surfactants and the like), pH/pH adjustment, particle sizes, enzymes (type and concentration/dosage), and feedstock, for example.

Still moving in a direction towards the bottom of the settling tank 102/slurry mixture and below the layer of (fine) starch and larger fibrous particles/fragments, a layer or portion of light “degermed” ground feedstock (e.g., degermed corn flour), which generally excludes free germ, as indicated generally by reference numeral 118, is present. Germ that may remain in this layer of the settling tank 102 is bound to other (more dense) corn flour particles and will not float freely. And then finally, the settled slurry mixture can define or include a bottom or lowermost portion of more dense or heavy “degermed” ground feedstock (e.g., corn flour), as indicated generally by reference numeral 120, relative to the adjacent lighter or less dense “degermed” corn flour. The more dense or heavy degermed corn flour 120 that has settled at or near the bottom of the settling tank 102 is understood to be desirable feed for producing “clean(er)” sugar streams, which may then be ultimately subjected to fermentation or other processes for the production of biochemicals because it can contain less oil, ash, fructose, endogenous enzymes, and other (e.g., soluble) impurities, as compared to the less dense or lighter degermed corn flour layer, for example.

With continuing reference to FIGS. 2 and 3, the settling tank 102 can be provided with a bottom discharge outlet 124 that can be situated at or near the bottom of the conical portion for initially removing the bottommost portion (e.g., the heavy degermed corn flour) therefrom to provide a degermed stream for producing a sugar (or carbohydrate) stream that can be subjected to fermentation or other processes for the production of a biochemical(s) (or other chemicals). In another example, the sugar (or carbohydrate) stream can be used as an ingredient, such as in a food product or pharmaceutical. The settling tank 102 also has another outlet 126 that can be situated at or near the top of the main or cylindrical portion for removing the top portion of the slurry 112, which can include the germ and/or germ fragments, therefrom.

Concerning the bottommost portion 120, a defined/desirable amount of mass/volume of the heavy degermed corn flour 120 initially can be removed via the bottom discharge outlet 124 of the settling tank 102 for further processing, which can include various biochemical processes such as fermentation or other processes and which may be performed offsite. In one example, the defined amount initially can be sent to a liquefaction step 128, indicated here as liquefaction I, not unlike the liquefaction step in FIG. 1, and further processed by means and methods in the art such as to produce alcohol (e.g., ethanol). In this example, the heavy degermed corn flour layer 120 may be further diluted prior to liquefaction, such as by adding cook water thereto. As indicated above, this degermed stream 120 can be desirable feed for producing “clean(er)” sugar streams because it can contain less oil, ash, fructose, endogenous enzymes, and other (e.g., soluble) impurities.

Next, a defined/desirable amount of mass/volume of the lighter degermed corn flour 118 similarly can be removed via the bottom discharge outlet of the settling tank 102 for further processing, which can include various biochemical processes, including fermentation or other processes and which may be performed offsite. The lighter degermed corn flour 118 can be sent or subjected, for example, to the more traditional ethanol system and method of FIG. 1, whereat the next processing step may be liquefaction 16, indicated here as liquefaction II, followed by the remaining steps, for example. The lighter or less dense degermed corn flour layer 118 may be further diluted prior to liquefaction, such as by adding cook water thereto. In one example, after the settling tank 102, the lighter degermed and heavy degermed corn flour streams 118 can be sent to separate biochemical processes for further processing. Still further, any additional layer(s) or liquid portion 114 and/or 116 thereafter optionally can be removed and sent, for example, along with the lighter degermed corn flour 118, to the same biochemical process(es), such as to liquefaction II, or a different process/liquefaction process altogether. This can help ensure that any fine/soluble starch can be converted, for example, to alcohol (e.g., to ethanol) so as to limit or reduce any yield loss.

In another embodiment, although not shown, a defined/desirable amount of mass/volume of the heavy degermed corn flour 120 and the lighter degermed corn flour 118 can be removed together via the bottom discharge outlet of the settling tank 102 for further processing, which can include various biochemical processes, including fermentation or other processes and which may be performed offsite. The heavier and lighter degermed corn flour 120, 118 can be sent or subjected together, for example, to liquefaction step 128, indicated here as liquefaction I, not unlike the liquefaction step in FIG. 1, and further processed by means and methods in the art such as to produce alcohol (e.g., ethanol), or sent to the more traditional ethanol system and method of FIG. 1, whereat the next processing step may be liquefaction 16, indicated here as liquefaction II, followed by the remaining steps, for example, or another process altogether. Similarly, in another example, the heavier and lighter degermed corn flour 120, 118 can be removed separately, as discussed above, and then recombined and subjected together to additional processing steps such as either liquefaction 1 or 2, or another process altogether.

To determine the defined/desirable amount of mass/volume to release from the settling tank 102, testing and trials can be performed as would be understood by those skilled in the art to determine amounts/cutoffs. In one example, testing of ash can be performed to evaluate or determine the mass/volume amount in the various layers, such as for the heavy and lighter degermed corn flour layers/streams 120, 118. While only one settling tank 102 is shown, it should be understood that more than one settling tank 102 may be utilized either in series or parallel.

With continuing reference to FIGS. 2 and 3, as indicated above, the upper or top/topmost portion or layer 112, which includes germ and/or germ fragments (e.g., fine germ) as well as red dog (red fibrous material), stalks, and other undesirable/unfermentable components, can be removed via the top outlet 126 situated at or near the top of the settling tank 102, such as via gravity and/or a pump. The upper layer 112, including the germ and/or germ fragments, can define a germ stream and be sent to an optional dewatering device (e.g., a screen) 130 whereat the germ and/or germ fragments and other unfermentable components can be separated from a liquid portion, which can be recombined with or recycled back to the cook water, as shown. In one example, to help facilitate the release of more germ and/or germ fragments, and/or grain oil, additives such as brine, surfactant, flocculant, enzyme, extractant, oil, chemicals, pH agents, or combinations thereof may be introduced after removal of the top portion 112 from the settling tank 102, such as prior to and/or during the optional dewatering device 130. Brine, surfactants, flocculants, enzymes, extractants, oil, pH agents, and chemicals can help coagulate oil and oil bodies together resulting in greater density separation through dewatering device 130, for example. The dewatering device 130 can include a screen, such as a gravity screen. In another example, the dewatering device 130 can include a paddle screen, pressure screen, and the like. The dewatering device 130 also can include multiple dewatering devices, which can be utilized in series or parallel. The screen of the dewatering device 130 can be provided with openings desirably sized to separate the germ and/or germ fragments from the liquid portion. In one example, the screen openings can be of a size from 50 micron to 2,000 micron, but some facilities can operate at less than or greater than these screen sizes.

From the settling tank 102 (or the optional dewatering device 130, if present), the germ stream may be subjected to an optional milling step 132, which can include one or more milling devices (e.g., a disc mill), in which the germ and/or germ fragments can be broken down or ground into smaller pieces or particles to release oil from the germ and/or germ fragments for recovery thereafter (directly or later in the process). In one example, the one or more disc mills may be replaced by a roller mill, hammer mill, pin mill, a homogenizer, or the like. The disc mill, roller mill, hammer mill, pin mill, homogenizer, and the like can be utilized in series or parallel. In one example, the disc mill can include a 36″ disc mill. In addition, it should be understood that the mill gap settings can be varied, as needed, to produce optimal settings, such as for the desired feedstock.

From the optional milling step 132, the now milled germ and/or germ fragments stream or portion, or the germ and/or germ fragments (along with the other undesirable unfermentable components) can be sent on for further processing, which similarly can include various biochemical processes, including fermentation or other processes. In one example, the germ and/or germ fragments (milled or not) can be sent or subjected to the more traditional ethanol process of FIG. 1 (or other related process), whereat the next processing step may be liquefaction 16, indicated here as liquefaction II. In one example, the germ stream (milled or not) may be combined with the lighter degermed corn flour 118 and further processed together. Alternatively or additionally, optionally milled germ and/or germ fragments, along with released or free oil, can be added to liquefaction I (along with the heavy degermed corn flour 120) or to the whole stillage (See, e.g., FIG. 1 after distillation and dehydration step 24) for further processing, which can include oil recovery/increased oil yield such as via an oil recovery centrifuge. Other processing systems of the optionally milled germ and/or germ fragments here can include further refinement such as to specifically produce a liquid vegetable oil stream (e.g., for refining into food grade corn oil) or use as a feed stock for further processing to a biochemical(s) (e.g., biodiesel and/or renewable diesel) or other chemicals. Oil recovery can be in addition to other oil recovery to increase overall oil yield.

As indicated above, the system and method 100 can provide multiple streams for producing multiple sugar (or carbohydrate) streams (of different compositions) such as for biochemical production, with said multiple sugar (or carbohydrate) streams being used to produce the same or different biochemicals, as discussed next. While the method and system 100 of FIGS. 2 and 3 for either the lighter degermed corn 118 (and optionally for the germ and/or germ fragments (milled or not)) and the heavier degermed corn flour 120 can provide for the production of alcohol, such as ethanol, it should be understood that other alcohols, e.g., butanol, as well as other biochemicals, can be produced here. In one example, other options for these layers, particularly the heavier degermed corn flour, which can produce a desirable/more desirable sugar (or carbohydrate) stream, aside from fermentation, can include further processing or refining of glucose to fructose or other simple or even complex carbohydrates, processing into feed, microbe-based fermentation (as opposed to yeast based) and other various chemical, pharmaceutical or nutraceutical processing (such as propanol, isobutanol, citric acid or succinic acid), and the like. Still further, the milling with front end germ separation described in detail in FIGS. 2 and 3 may be incorporated into other methods and systems that can produce additional or more desirable coproducts, along with DDG(S) and DWG(S). And as indicated above, the milling with front end germ separation described in detail in FIGS. 2 and 3 may be incorporated into any traditional, modified traditional, or non-traditional dry grind process/plant and by doing so may help keep overall capital costs at a desirable level for the dry grind plant.

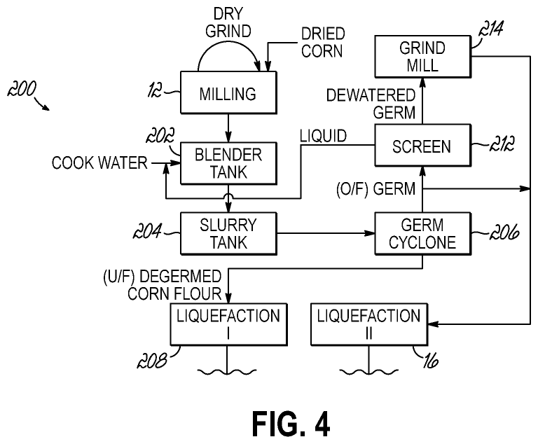

With reference now to FIG. 4, another embodiment of a system and method with front end germ separation for producing/ultimately producing one or more sugar (or carbohydrate) streams such as for biochemical production (e.g., alcohol), collectively numeral 200, is shown as a variation of FIG. 2 above, which is generally based on the dry grind process 10 of FIG. 1 and described hereinabove. In one example, the sugar (or carbohydrate) stream(s) may be produced offsite. In another example, a carbohydrate stream(s) initially may be produced after front end germ separation such as for biochemical production. In another example, a carbohydrate stream(s) initially may be produced after front end germ removal followed by production of a sugar stream(s) such as for biochemical production. This dry grind system and method 200, similar to the above system and method 100, can reduce the amount of undesirable, unfermentable components, such as germ/germ fragments, prior to fermentation (or other reaction process), thereby helping to keep overall capital and operational costs at a desirable level for the dry grind plant and producing a more desirable sugar (or carbohydrate) stream, such as for fermentation or other reaction processes or utilized directly as an ingredient (such as in a food product or pharmaceutical, for example). As with FIGS. 2 and 3, it is noted that certain reference numerals used in FIG. 1 are used here to represent like devices and/or steps in the system and method 200 of FIG. 4.

In this embodiment, the grain (e.g., corn) feedstock, which may be dried, initially can be milled at milling step 12, as discussed above, with the ground meal (corn flour) being mixed with liquid, such as cook water, at a blender tank 202 to create a slurry. In one example, the cook water can be backset and/or fresh (or clean) water and can be piped in by means and methods known in the art, such as via a pump or other methods. In one example, the temperature of the liquid (e.g., cook water) can be from about 60° F. to about 220° F. In another example, the temperature of the liquid (e.g., cook water) can be from about 110° F. to about 150° F. In another example, the temperature of the liquid (e.g., cook water) can be about 130° F. The temperature of the liquid here may be increased or maintained with the assistance of an eductor (not shown) or other like device to attain or maintain the desired temperature. In one example, the eductor may be situated before or after the blender tank 202 and can receive the incoming cook water or the outgoing slurry stream.

Initially, the ground feedstock (e.g., corn flour) can be transported by means and methods known in the art, such as via a conveyor and the like, to the blender tank 202. The blender tank 202 can be generally cylindrical in shape (although various other shapes are contemplated and known in the art) and can be equipped with at least one agitator (like agitator 106a, 106b, as shown and described above in FIG. 3), which can include one or more rotating fans/paddles or blades, controlled by a motor as known in the art. In one example, the agitator can define a screw such as a helical screw. The agitator can include one or more fans/paddles or blades, as desired/needed, and can be of any desired shape or size. The agitator can be stopped and started, as desired, to mix (e.g., homogenously mix) the ground feedstock and liquid, e.g., cook water, to a desired or targeted dry solids (DS) level such as where the slurry is pumpable and still able to be agitated. In one example, the desired DS of the slurry in the blender tank 202 can be from about 25% to 36%. In another example, the desired DS of the slurry can be from about 32% to 34%. In one example, the blender tank 202 can be operated on level control, increasing or decreasing the amount of material moved or pumped out of the blender tank 202 to match the amount of material entering, by means and methods known in the art. While only one blender tank 202 is shown, it should be understood that more than one blender tank 202 may be utilized either in series or parallel.

With continuing reference to FIG. 4, the blender tank 202 can include one or more liquid inlets that communicate with an interior of the blender tank 202 to fill the tank with liquid (e.g., wash water). One or more enzymes can be added to the slurry here. In one example, the one or more enzymes can include amylase, alpha-amylase, glucoamylase, fungal, phytase, protease (e.g., serine protease), cellobiose, cellulase, hemicellulase, xylanase, glucanase, beta-glucanase, transglutaminase (e.g. microbial), and the like.

The slurry mixture can be piped/sent from the blender tank 202 to an optional slurry tank 204 to assist with complete hydration of the corn flour, such as after a pre-determined amount of time, which can be identified based upon testing of the stream/slurry mixture and reaching a desired DS level. From the optional slurry tank 204 or the blender tank 202, the slurry mixture, which includes a desired DS level and has lighter (or less dense) germ/germ fragments (fine germ) and other lighter or fine particles (e.g., red dog, stalks, and free oil) along with more dense material/particles (e.g., starch, protein, and fiber), can be piped/sent to one or more germ cyclones 206, as are known in the art, to separate the slurry mixture/stream into overs or an overflow (O/F) and unders or an underflow (U/F). The overs include lighter, less dense material and define a germ stream, including germ and/or germ fragments (along with red dog (red fibrous material), stalks, and other undesirable/unfermentable components) and the unders includes more dense material and defines a “degermed” feedstock (e.g., corn flour) stream, including protein, starch, and fiber. The more dense or heavy degermed corn flour is understood to be desirable feed for producing “clean(er)” sugar streams, which may then ultimately be subjected to fermentation or other processes for the production of biochemicals because the sugar stream can contain less ash, fructose, endogenous enzymes, and other (e.g., soluble) impurities. To help facilitate the release of more germ and/or germ fragments, and/or grain oil, additives such as brine, surfactant, flocculant, enzyme, extractant, oil, chemicals, pH agents, or combinations thereof may be introduced prior to and/or during use of the germ cyclone(s). Brine, surfactants, flocculants, enzymes, extractants, oil, pH agents, and chemicals can help coagulate oil and oil bodies together resulting in greater density separation through the germ cyclone 206, for example.

The germ cyclone 206 utilizes centrifugal and pressure differential type forces to separate the lighter density germ and/or germ fractions (along with red dog, stalks, free oil, and other lighter weight constituents) from the remaining corn flour material, such as the protein, starch, and fiber, in the slurry. The germ and/or germ fractions contain the majority of the grain oil and thus has a lower density compared to the other grain components, such as fiber, protein, and starch. The germ cyclone 206 uses pressure from the feeding slurry stream to create a centrifuge type flow within the conical shaped cyclone device, where lighter, less dense materials (i.e., germ or germ fraction) will tend to gravitate to the top of the conical device and the heavier more dense materials will come out the bottom of the germ cyclone 206. More specifically, the flow into the germ cyclone 206 enters tangentially into a barrel of the germ cyclone 206. The centrifugal force of the slurry circling the barrel of the germ cyclone 206 forces the denser particles of protein, starch, and fiber to an outer wall of the germ cyclone 206 while the less dense germ fragments and free oil droplets collect in a center of the germ cyclone 206. The hydraulic flow entering the germ cyclone 206 displaces the light germ particles and oil and they exit a light phase port in a top center of the germ cyclone 206. The denser protein, starch, and fiber particles that have been collected near the wall of the germ cyclone 206 settle due to gravity and exit out a bottom heavy phase port of the germ cyclone 206. The germ cyclone 206 may have a control valve on the discharge of either the overflow (lighter dense flow) or underflow (heavier dense flow) that controls the pressure and flow rate out of the device. In addition, although a single germ cyclone is depicted, it should be understood that a plurality of germ cyclones may be situated in-line, either in series and/or parallel. One such suitable germ cyclone 206 is the GS6″ model available from Fluid Quip, Inc. of Springfield, Ohio.

Continuing with the flow from the germ cyclones 206, the degermed corn flour stream can be sent on for further processing, which can include various biochemical processes such as fermentation or other processes. In one example, the degermed corn flour stream can be sent to a liquefaction step 208, indicated here as liquefaction I, such as not unlike the liquefaction step in FIG. 1, and further processed by means and methods in the art such as to produce alcohol (e.g., ethanol) from one or more sugar (or carbohydrate) streams. In this example, the heavy degermed corn flour layer may be further diluted prior to liquefaction, such as by adding cook water thereto. As indicated above, this degermed stream can be desirable feed for producing “clean(er)” sugar (or carbohydrate) streams because it can contain less ash, fructose, endogenous enzymes, and other (e.g., soluble) impurities.

With respect to the overs (O/F) from the germ cyclone(s) 206, the germ stream (including germ fragments, red dog (red fibrous material), stalks, and other undesirable/ unfermentable components) can be optionally sent to a dewatering device (e.g., a screen) 212 whereat the germ and/or germ fragments and other unfermentable components can be separated from a liquid portion, which can be recombined with or recycled back to the cook water, as shown. The dewatering device 212 can include a screen, such as a gravity screen. In another example, the dewatering device 212 can include a paddle screen, pressure screen, and the like. The dewatering device 212 also can include multiple dewatering devices, which can be utilized in series or parallel. The screen of the dewatering device 212 can be provided with openings desirably sized to separate the germ and/or germ fragments from the liquid portion. In one example, the screen openings can be of a size from 50 micron to 2,000 micron, but some plants can operate at less than or greater than these screen sizes.

From the germ cyclone 206 (or the optional dewatering device 212, if present), the germ stream may be subjected to an optional milling step 214, which can include one or more milling devices (e.g., a disc mill), in which the germ and/or germ fragments can be broken down or ground into smaller pieces or particles to release oil from the germ and/or germ fragments for recovery thereafter (directly or later in the process). In one example, the one or more disc mills may be replaced by a roller mill, hammer mill, pin mill, a homogenizer, or the like. The disc mill, roller mill, hammer mill, pin mill, homogenizer, and the like can be utilized in series or parallel. In one example, the disc mill can include a 36″ disc mill. In addition, it should be understood that the mill gap settings can be varied, as needed, to produce optimal settings, such as for the desired feedstock.

From the optional milling step 214, the now milled germ stream, or, if not milled, the germ and/or germ fragments (along with the other undesirable unfermentable components) can be sent on for further processing, which similarly can include various biochemical processes, including fermentation or other processes. In one example, the germ and/or germ fragments (milled or not) can be sent or subjected to the more traditional ethanol process of FIG. 1 (or other related process), whereat the next processing step may be liquefaction 16, indicated here as liquefaction II. In another example, a portion of the germ stream may be combined with the milled germ stream, as shown, prior to further processing. Alternatively or additionally, optionally milled germ and/or germ fragments can be added to the whole stillage (See, e.g., FIG. 1 after distillation and dehydration step 24) for further processing. Other processing systems of the optionally milled germ and/or germ fragments here can include further refinement such as to produce a liquid vegetable oil stream (e.g., for refining into food grade corn oil) or use as a feed stock for further processing to a biochemical(s) (e.g., biodiesel and/or renewable diesel) or other chemicals. In another example, although not shown, the optionally milled germ and/or germ fragments can be sent or subjected, for example, to liquefaction step 208, indicated here as liquefaction I, not unlike the liquefaction step in FIG. 1, and further processed along with the degermed corn flour by means and methods in the art such as to produce alcohol (e.g., ethanol) and for optional oil recovery, such as via an oil recovery centrifuge.

As indicated above, the system and method 200 can provide multiple streams for producing multiple sugar (or carbohydrate) streams (of different compositions) such as for biochemical production, with said multiple sugar (or carbohydrate) streams being used to produce the same or different biochemicals, as discussed next. While the method and system 200 of FIG. 4 for either the germ (and/or germ fragments) stream (milled or not), and the heavier degermed corn flour stream can provide for the production of alcohol, such as ethanol, it should be understood that other alcohols, e.g., butanol, as well as other biochemicals, can be produced here. In one example, other options for these streams, particularly the heavier degermed corn flour stream, which can produce a desirable/more desirable sugar (or carbohydrate) stream, aside from fermentation, can include further processing or refining of glucose to fructose or other simple or even complex carbohydrates, processing into feed, microbe-based fermentation (as opposed to yeast based) and other various chemical, pharmaceutical or nutraceutical processing (such as propanol, isobutanol, citric acid or succinic acid), and the like. Still further, the milling with front end germ separation described in FIG. 4 may be incorporated into other methods and systems that can produce additional or more desirable coproducts, along with DDG(S) and DWG(S). In the end, the milling with front end germ separation described in detail in FIG. 4 may be incorporated into any traditional, modified traditional, or non-traditional dry grind process/plant and by doing so may help keep overall capital costs at a desirable level for the dry grind plant.

With the system and method 200, as shown in FIG. 4, when the degermed corn flour has been subjected to liquefaction and saccharification, like discussed above in FIG. 1, beneficial characteristics have been shown when compared to non-degermed corn flour that has been subjected to the same liquefaction and saccharification. In one example, comparative testing (degermed stream vs. non-degermed stream) of a final saccharified product showed 54% reduction in fructose, 37% reduction in conductivity, and a 0.8% increase in dextrose, i.e., from 94.6% to a 95.4%. The final saccharified product also contained 28% less ash when using the degermed corn flour.

While the present invention has been illustrated by the description of one or more embodiments thereof, and while the embodiments have been described in considerable detail, they are not intended to restrict or in any way limit the scope of the appended claims to such detail. For example, this technology also has the potential to reduce mycotoxin levels in protein products produced in a dry grind process. The various features shown and described herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of Applicant's general inventive concept.

What is claimed is:

1. A method with front end germ separation for producing a sugar stream, comprising:
mixing ground grain particles derived from a feedstock of grain and/or grain components with a liquid within a settling tank to produce a slurry that comprises starch, fiber, and germ particles;
via weights, separating out lighter germ particles from the slurry in the settling tank to degerm the slurry and allowing heavier solids within the slurry to settle out within the settling tank;
removing a heavier first portion of the settled solids followed by removing a lighter second portion of the settled solids from the settling tank;
subjecting the heavier first portion and optionally the lighter second portion to liquefaction to provide a liquefied starch solution, which includes starch and fiber; and
subjecting the liquefied starch solution to saccharification to convert the starch to simple sugars thereby producing a sugar stream.

2. The method of claim 1 wherein subjecting the heavier first portion and optionally the lighter second portion to liquefaction to provide a liquefied starch solution comprises subjecting the heavier first portion and the lighter second portion separately to a first liquefaction and a second liquefaction to provide a first and a second liquefied starch solution, with each including starch and fiber; and subjecting the first and second liquefied starch solutions to saccharification to convert the starch to simple sugars thereby producing a sugar stream.

3. The method of claim 2 wherein the first and second liquefaction are different liquefaction steps.

4. The method of claim 2 wherein subjecting the first and second liquefied starch solutions to saccharification to convert the starch to simple sugars comprises subjecting the first and second liquefied starch solutions separately to a first and a second saccharification to convert the starch to simple sugars thereby producing a first and second sugar stream.

5. The method of claim 4 wherein the first and second saccharification are different saccharification steps.

6. The method of claim 1 wherein subjecting the heavier first portion and optionally the lighter second portion to liquefaction comprises subjecting the heavier first portion and the lighter second portion to liquefaction to provide a liquefied starch solution, which includes starch and fiber and wherein the heavier first portion and the lighter second portion are subjected to the same liquefaction step.

7. The method of claim 7 wherein subjecting the heavier first portion and the lighter second portion to liquefaction to provide a liquefied starch solution comprises subjecting the heavier first portion and the lighter second portion separately to a first liquefaction and a second liquefaction to provide a first and a second liquefied starch solution, with each including starch and fiber, and subjecting the first and second liquefied starch solutions separately to a first and a second saccharification to convert the starch to simple sugars thereby producing a first and second sugar stream.

8. The method of claim 1 further comprising dewatering the separated germ particles and grinding the dewatered germ particles to release oil.

9. The method of claim 8 further comprising subjecting the lighter second portion and the ground dewatered germ particles together to the second liquefaction to provide the second liquefied starch solution, which includes starch, fiber, ground germ particles, and the released oil.

10. The method of claim 7 further comprising subjecting the simple sugars of the first sugar stream and the simple sugars of the second sugar stream to a biochemical process to produce a biochemical.

11. The method of claim 10 wherein the biochemical is ethanol.

12. The method of claim 10 wherein the biochemical process is fermentation.

13. The method of claim 10 wherein the biochemical process includes a catalytic or chemical reaction.

14. The method of claim 1 wherein subjecting the simple sugars of the first sugar stream and the simple sugars of the second sugar stream to a biochemical process to produce a biochemical comprises subjecting the simple sugars of the first sugar stream and the simple sugars of the second sugar stream to separate first and second biochemical processes, respectively, to produce different biochemicals.

15. The method of claim 1 further comprising diluting the heavier first portion and/or the lighter second portion of the settled solids prior to liquefaction.

16. The method of claim 1 wherein the desired dry solids of the slurry in the settling tank is from about 30% to 36%.

17. The method of claim 1 wherein mixing of the ground grain particles with the liquid with the liquid within the settling tank occurs in batches or a semi-continuous type manner.

18. The method of claim 1 wherein the slurry is allowed to settle for about 30 seconds to about 60 minutes.

19. The method of claim 1 wherein removing a heavier first portion of the settled solids followed by removing an optional lighter second portion of the settled solids from the settling tank comprises removing a defined amount of mass/volume of settled solids to define the heavier first portion followed by removing a defined amount of mass/volume of remaining settled solids to define the lighter second portion.

20. A method with front end germ separation for producing a sugar stream, comprising:
mixing ground grain particles derived from a feedstock of grain and/or grain components with a liquid within a settling tank to produce a slurry that comprises starch, fiber, and germ particles;
via weights, separating out lighter germ particles from the slurry in the settling tank to degerm the slurry and allowing heavier solids within the slurry to settle out within the settling tank;
removing a defined amount of mass/volume of the settled solids from the settling tank to provide a heavier first portion followed by removing a defined amount of mass/volume of remaining settled solids from the settling tank to provide a lighter second portion;
subjecting the heavier first portion to a first liquefaction and separately subjecting the lighter second portion to a second liquefaction to provide a first and a second liquefied starch solution, respectively, which include starch and fiber; and
subjecting the first liquefied starch solution to a first saccharification and separately subjecting the second liquefied starch solution to a second saccharification to convert the starch to simple sugars in the first and second liquefied starch solutions thereby producing a first sugar stream and a second sugar stream.

21. A system with front end germ separation for producing a sugar stream, comprising:
a settling tank within which ground grain particles derived from a feedstock of grain and/or grain components mix with a liquid to produce a slurry that comprises starch, fiber, and germ particles, the settling tank configured to separate out lighter germ particles, via weights, from the slurry to degerm the slurry, and in which heavier solids within the slurry settle out, the settling tank further configured to remove a heavier first portion of the settled solids followed by an optional lighter second portion of the settled solids from the settling tank;
a liquefaction system that receives the heavier first portion and optionally the lighter second portion to provide a liquefied starch solution, which includes starch and fiber, and whereat the starch begins to convert to oligosaccharides; and
a saccharification system that is situated after the liquefaction system and that receives the liquefied starch solution, the saccharification system converts the oligosaccharides to simple sugars thereby producing a sugar stream.
